(19) United States
(12) Patent Application Publication
(10) Pub. No.: US 2009/0111906 A1
(43) Pub. Date: Apr. 30, 2009

(54) Use of Dispersants to Concentrate Mineral Matter in Water, Dispersions Obtained and Their Uses

(76) Inventors: Christian Jacquemet, Lyon (FR); Jacques Mongoin, Quincieux (FR); Jean-Marc Suau, Lucenay (FR)

Correspondence Address: AMSTER, ROTHSTEIN & EBENSTEIN LLP, 90 PARK AVENUE, NEW YORK, NY, 10016, US

(21) Appl. No.: 12/084,862

(22) PCT Filed: Dec. 13, 2006
(86) PCT No.: PCT/IB2006/003681

§ 371 Date: May 12, 2008

(30) Foreign Application Priority Data
Dec. 20, 2005 (FR) 0512928

(51) Int. Cl. A61K 8/81; C08K 11/00; C08K 3/26; C08K 3/16; D21H 17/66; C08K 3/34; C08K 3/28; C08K 3/22

(52) U.S. Cl. 523/105; 524/80; 524/425; 524/438; 524/443; 524/428; 524/436; 162/181.2

(57) ABSTRACT

The invention consists in the use of dispersing agents, in a process of manufacture of an aqueous dispersion of mineral matter, by:
a) grinding with a small amount of dry extract (≦40%) of the said mineral matter in water without a dispersing agent and/or grinding aid agent,
b) concentration with a large amount of dry extract (≧65%) by mechanical and/or thermal means,
   characterised in that:
at least one dispersing agent is introduced between stage a) and stage b), and/or during stage b), and/or during and after stage b).
in the form of a combination:
   of at least one homopolymer of acrylic acid,
   and of at least one fluorinated mineral compound.

The invention relates firstly to a use of new dispersing agents enabling mineral matter to be concentrated in water.

It also concerns the aqueous dispersions of mineral matter then obtained, together with their uses in aqueous formulations containing mineral matter, such as calcium carbonate, and notably in the paper fields and more specifically in connection with the manufacture of paper sheet and of paper sheet coating, paint, plastic and cosmetics, such as notably in the manufacture of dental pastes.

The skilled man in the art who is a manufacturer of aqueous suspensions and dispersions of mineral matter, such as notably calcium carbonate, has for a very long time been familiar with the use of dispersion agents and/or grinding aid agents with an acrylic homopolymer and/or copolymer base, with a view to maintaining the said mineral matter in suspension in water at high dry matter concentrations, in a stable manner over time.

They are thus familiar with patents FR 2 603 042, EP 0 100 947, EP 0 127 388, EP 0 129 329 and EP 0 542 644, which describe the use for the above-mentioned purposes of such polymers, totally or partially neutralised by various neutralisation agents, and having a low molecular weight.

In connection with these same applications, they are also familiar with patents FR 2 488 814, EP 0 100 948 and EP 0 542 643, which teach the use of the fraction of acrylic homopolymers and/or copolymers, the specific viscosity of which is between 0.3 and 0.8, as measured by the common method described in the patents concerned.

Nevertheless, these various solutions which enable aqueous suspensions of mineral matter stable over time to be obtained do not enable mineral particles such as notably calcium carbonate to be dispersed in water, with high dry weight contents of mineral matter (greater than or equal to 65% of the total weight of the dispersion) when the latter are derived from the following process:
a) grinding of mineral matter in an aqueous medium without the use of a dispersing agent and/or grinding aid agent, and at a low dry matter concentration (with the dry extract, or the dry weight content of mineral matter then being less than 40% relative to the total weight of the suspension),
b) followed by mechanical and/or thermal concentration, with a view to obtaining aqueous dispersions of mineral matter having a dry matter content greater than or equal to 65%, relative to the total weight of the dispersion,
   where a dispersing agent is introduced between stage a) and stage b), and/or during stage b), and/or during and after stage b).

In the remainder of the application, the Applicant may make reference to such a process through the expression “process of grinding with a small amount of dry extract without dispersing agent followed by concentration with a large amount of dry extract in the presence of a dispersing agent”.

With a view to resolving this particular technical problem, the skilled man in the art is familiar with document EP 0 027 996, which describes a process for manufacture of aqueous suspensions of mineral matter by a stage of grinding in a wet medium without dispersing agent, the suspension then being filtered, and the filter cake then obtained is dried or transformed through the addition of a dispersing agent into a suspension of low viscosity; the grinding stage is undertaken at a dry matter concentration of less than 60% by total weight of the suspension, and the final suspension obtained after addition of the dispersing agent has a dry matter content of at least more than 80% of its total weight.

But if the skilled man in the art does not choose appropriately the dispersing agent which they use, they do not succeed in obtaining sufficiently high final dry matter contents whilst continuing to maintain the handleable character for the suspension obtained; and nothing is taught to the skilled man in the art concerning the choice of particular dispersing agents in this document EP 0 027 996, nor concerning the experimental conditions to be used to obtain such high dry matter contents, and viscosities compatible with the use of such a suspension: such a document does not therefore enable them to resolve the technical problem of the present Application.

It is precisely for this reason that 2 other documents, subsequent to document EP 0 027 996, have addressed the same technical problem, but from the standpoint of the choice of particular dispersion agents, which enable the technical problem of the present Application to be resolved effectively. We shall see in the remainder that these documents are not devoid of drawbacks for the skilled man in the art.

Thus, the skilled man in the art is familiar with patent WO 01/048 093, which teaches the use of homopolymers and copolymers of acrylic acid with different allylic and vinylic water-soluble monomers, of a molecular weight with a viscosity index ranging from 0.08 to 0.80 according to the method described in the patent application concerned.

Patent EP 0 850 685 teaches them another solution which consists in using copolymers of acrylic acid and of maleic acid, having a molar ratio between these two units of between 2:1 and 10:1, and an average molecular mass of between 1,000 and 100,000 Daltons.

Finally, the skilled man in the art is also familiar with document EP 1 147 061, which describes a process similar to the above-mentioned one, but nonetheless different. This document describes a process characterised by the stages of manufacture of a diluted aqueous suspension of carbonate, the content by weight of which does not exceed 40%, followed by elimination of water to obtain a content by weight of between 45% and 65%, followed by possible addition of a dispersing agent, followed by subsequent elimination of water under reduced pressure to increase the content by weight by at least 5%, finally followed by mechanical treatment of the suspension obtained. This process is thus different from that forming the subject of the present Application; moreover it appears more complex to implement, because of the higher number of stages, and the need to have apparatus allowing work under reduced pressure.

The skilled man in the art has now developed a new solution with a view to obtaining aqueous dispersions of mineral particles such as, notably, calcium carbonate, having a large amount of dry extract (higher than 65% by weight of mineral matter relative to the total weight of the dispersion) and an immediate Brookfield™ viscosity measured at 100 RPM of less than 5,000 mPa·s, during a stage of mechanical and/or thermal concentration, following a stage of grinding with a low dry matter concentration (less than 40% by weight of mineral matter relative to the total weight of the dispersion), in an aqueous medium, and without the use of a dispersing agent and/or grinding aid agent, which was proposed by none of the documents of the prior art, except for documents WO 01/048 093 and EP 0 850 685.

In addition, documents WO 01/048 093 and EP 0 850 685 teach, among other things, that homopolymers and/or copolymers of acrylic acid, totally or partially neutralised, are not suitable to disperse calcium carbonate in water using the process of grinding with a small amount of dry extract without a dispersing agent followed by concentration with a large amount of dry extract in the presence of a dispersing agent. This teaching is taken as being except for the very particular homopolymers and copolymers of acrylic acid which are the subject of the selected inventions described in documents WO 01/048 093 and EP 0 850 685. The very restrictive character of the solutions does not give a high degree of latitude to the skilled man in the art in the choice of the dispersants which they wish to use.

And, in a completely surprising manner, the present invention enables the skilled man in the art to use homopolymers of acrylic acid, without any restriction concerning them. And, unexpectedly, the use according to the invention of homopolymers of acrylic acid in combination with a fluorinated mineral compound precisely enables the skilled man in the art to disperse calcium carbonate according to the process of grinding with a small amount of dry extract without dispersing agent followed by concentration with a large amount of dry extract in the presence of a dispersing agent. The skilled man in the art then succeeds in obtaining, in a surprising manner, bearing in mind notably the teaching given by documents WO 01/048 093 and EP 0 850 685, high dry extracts (greater than 65% by weight of mineral matter relative to the total weight of the dispersion) and a low immediate Brookfield™ viscosity measured at 100 RPM (less than 5,000 mPa·s).

Lastly, the present invention enables the skilled man in the art to obtain, by optimisation of the pair formed by the homopolymer of acrylic acid and of the fluorinated mineral compound, of solutions which prove, in a surprising manner, even more effective than those proposed in documents WO 01/048 093 and EP 0 850 685; some of the solutions described in the present Application indeed enable the skilled man in the art to obtain very high dry extracts (greater than 70% by weight of mineral matter relative to the total weight of the dispersion) and a very low immediate Brookfield™ viscosity measured at 100 RPM (less than 500 mPa·s and sometimes even less than 250 mPa·s), which is not revealed in the two documents WO 01/048 093 and EP 0 850 685.

The present invention is thus based on a use of dispersing agents in a process of manufacture of an aqueous dispersion of mineral matter, comprising the stages of:
a) preparation of an aqueous suspension of mineral matter by grinding of the said mineral matter in water without a dispersing agent and/or grinding aid agent, where the said suspension has a dry weight concentration of mineral matter less than or equal to 40% of its total weight,
b) concentration of the aqueous suspension of mineral matter obtained during stage a) by mechanical and/or thermal means, with a view to obtaining an aqueous dispersion of mineral matter of which the concentration by dry weight of mineral matter is at least equal to 65% of the total weight of the said dispersion,
   characterised in that:
at least one dispersing agent is introduced between stage a) and stage b), and/or during stage b), and/or during and after stage b).
in the form of a combination:
   of at least one homopolymer of acrylic acid,
   and of at least one fluorinated mineral compound.

In the field of aqueous dispersions of calcium carbonate, the Applicant is familiar with the use of fluorinated mineral compounds.

Thus, document U.S. Pat. No. 3,179,493 teaches the manufacture of a precipitated calcium carbonate, which is finely divided and of high purity, by reaction between a calcium salt and a carbonated compound, in the presence of a fluorinated compound chosen from among potassium, sodium and ammonium fluoride and silicofluoride. As for document U.S. Pat. No. 3,793,047, it teaches the surface treatment of a calcium carbonate by fluorinated compounds (H₂SiF₆ and MgSiF₆), with a view to obtaining opalescent particles which are resistant to abrasion and to acids.

Firstly, these patents are very far removed from the current problem of the skilled man in the art, since the technical problems covered are very different from the one mentioned in the present document. Secondly, in terms of the solutions adopted, the processes described in these two documents differ fundamentally from that of the present invention, since they are a process for manufacture of a calcium carbonate (U.S. Pat. No. 3,179,493) and a process for treatment of calcium carbonate (U.S. Pat. No. 3,793,047). Finally, the solutions used in these two documents also differ from that of the present invention, since they do not reveal the combination of a fluorinated mineral compound with a homopolymer of acrylic acid.

Therefore, the skilled man in the art has developed, in a surprising manner, a new solution with a view to dispersing in water, with a dry weight content of mineral matter greater than or equal to 65% by weight of the said dispersion and an immediate Brookfield™ viscosity measured at 100 RPM of less than 5,000 mPa·s, mineral particles such as notably calcium carbonate, derived from the process of grinding with a small amount of dry extract without dispersing agent followed by concentration with a large amount of dry extract in the presence of a dispersing agent.

This solution is thus based on a use of dispersing agents in a process of manufacture of an aqueous dispersion of mineral matter, comprising the stages of:
(a) preparation of an aqueous suspension of mineral matter by grinding of the said mineral matter without a dispersing agent and/or grinding aid agent, where the said suspension has a dry weight concentration of mineral matter less than or equal to 40% of its total weight,
(b) concentration of the aqueous suspension of mineral matter obtained during stage a) by mechanical and/or thermal means, with a view to obtaining a concentration by dry weight of mineral matter at least equal to 65% of the total weight of the said dispersion,
   characterised in that:
at least one dispersing agent is introduced between stage a) and stage b), and/or during stage b), and/or during and after stage b).
in the form of a combination:
   of at least one homopolymer of acrylic acid,
   and of at least one fluorinated mineral compound.

The means of mechanical and thermal concentration used during stage b) are chosen from among those well known to the skilled man in the art.

The skilled man in the art may also use, at the time of their choice—i.e. between stage a) and stage b), and/or during stage b), and/or during and after stage b)—every other dispersing agent of the prior art, with a view to optimising the characteristics of the final dispersion of mineral matter which they wish to obtain.

The use of dispersing agents according to the invention is characterised in that the aqueous suspension of mineral matter obtained during stage a) has a concentration by dry weight of mineral matter preferentially less than 35%, and more preferentially less than 30%, and in that the aqueous dispersion of mineral matter obtained during stage b) has a concentration by dry weight of mineral matter preferentially greater than 68%, and more preferentially greater than 70%.

This use is also characterised in that the aqueous dispersion of mineral matter obtained immediately after stage b) has a Brookfield™ viscosity measured at 100 RPM of less than 5,000 mPa·s, preferentially less than 2,000 mPa·s, very preferentially less than 1,000 mPa·s, and extremely preferentially less than 500 mPa·s.

The use of dispersing agents according to the invention is also characterised in that the mineral matter is chosen from among natural or synthetic calcium carbonate, the dolomites, kaolin, talc, cement, gypsum, lime, magnesia, titanium dioxide, satin white, aluminium trioxide, or again aluminium trihydroxide, the silicas, mica and a blend of these fillers one with another, such as talc-calcium carbonate blends, calcium carbonate-kaolin blends, or again blends of calcium carbonate with aluminium trihydroxide or aluminium trioxide, or again blends with synthetic or natural fibres, or again mineral costructures such as talc-calcium carbonate costructures or talc-titanium dioxide costructures, or their blends.

Preferentially they are chosen from among the natural or synthetic calcium carbonates or talc or kaolin or their blends.

In a very preferential manner they are chosen from among the natural or synthetic calcium carbonates or their blends.

The use of dispersing agents according to the invention is also characterised in that the fluorinated mineral compound on the one hand, and the homopolymer of acrylic acid on the other hand, are introduced simultaneously, or in that the fluorinated mineral compound is firstly introduced, and subsequently the homopolymer of acrylic acid, or firstly the homopolymer of acrylic acid, and subsequently the fluorinated mineral compound.

The use of dispersing agents according to the invention is also characterised in that the fluorinated mineral compound and the homopolymer of acrylic acid are introduced in the form of an aqueous suspension and/or an aqueous solution when they are introduced simultaneously.

The use of dispersing agents according to the invention is also characterised in that the fluorinated mineral compound is introduced in the form of powder and/or in the form of an aqueous suspension and/or in the form of an aqueous solution, and in that the homopolymer of acrylic acid is introduced in the form of an aqueous solution when these two compounds are introduced one after the other, whatever the order of introduction.

The use of dispersing agents according to the invention is also characterised in that between 0.1% and 3.0%, and preferentially between 0.5% and 1.5%, by dry weight relative to the dry weight of mineral matter, of at least one homopolymer of acrylic acid is used.

The use of dispersing agents according to the invention is also characterised in that between 0.01% and 0.5%, and preferentially between 0.05% and 0.25%, by dry weight relative to the dry weight of mineral matter, of at least one fluorinated mineral compound is used.

The use of dispersing agents according to the invention is also characterised in that the fluorinated mineral compound is chosen from among the compounds NaF, NaHF₂, H₂SiF₆, HKF₂, FeF₂, PbF₂, HNH₄F₂ and their blends, and preferentially from among the compounds NaF, H₂SiF₆, HKF₂, and their blends, and in that it is preferentially the compound NaF.

The use of dispersing agents according to the invention is also characterised in that the homopolymer of acrylic acid used is neutralised, totally or partially, by a neutralisation agent chosen from among the hydroxides and/or oxides of calcium or magnesium, the hydroxides of sodium, potassium or ammonium, or their blends, preferentially by a neutralisation agent chosen from among sodium or ammonium hydroxide, or their blends, and very preferentially by a neutralisation agent which is ammonium hydroxide.

The use of dispersing agents according to the invention is also characterised in that the homopolymer of acrylic acid used has an average molecular mass of between 1,000 and 150,000 Daltons, preferentially between 5,000 and 100,000 Daltons, and more preferentially between 15,000 and 80,000 Daltons.

The use of dispersing agents according to the invention is also characterised in that the homopolymer of acrylic acid has a neutralisation rate, expressed as a molar percentage of neutralised acid sites, of between 10 and 100 Daltons, preferentially between 50 and 100 Daltons, and more preferentially between 70 and 100.

Another object of the invention lies in the aqueous dispersions of mineral matter obtained by use according to the invention, in a manufacturing process, comprising the stages of:
(a) preparation of an aqueous suspension of mineral matter by grinding of the said mineral matter without a dispersing agent and/or grinding aid agent, where the said suspension has a dry weight concentration of mineral matter less than or equal to 40% of its total weight,
(b) concentration of the aqueous suspension of mineral matter obtained during stage a) by mechanical and/or thermal means, with a view to obtaining a concentration by dry weight of mineral matter at least equal to 65% of the total weight of the said dispersion,
   in the presence of at least one dispersing agent introduced between stage a) and stage b), and/or during stage b), and/or during and after stage b), where the said agent is introduced in the form of a combination:
of at least one homopolymer of acrylic acid,
and of at least one fluorinated mineral compound.

Another object of the invention is based on aqueous dispersions of mineral matter, characterised in that they contain:
at least one homopolymer of acrylic acid,
and at least one fluorinated mineral compound.

These aqueous dispersions of mineral matter are also characterised in that they have a concentration by dry weight of mineral matter preferentially greater than 68%, and more preferentially greater than 70%.

These aqueous dispersions of mineral matter are also characterised in that the mineral matter is chosen from among natural or synthetic calcium carbonate, the dolomites, kaolin, talc, cement, gypsum, lime, magnesia, titanium dioxide, satin white, aluminium trioxide, or again aluminium trihydroxide, the silicas, mica and a blend of these fillers one with another, such as talc-calcium carbonate blends, calcium carbonate-kaolin blends, or again blends of calcium carbonate with aluminium trihydroxide or aluminium trioxide, or again blends with synthetic or natural fibres, or again mineral costructures such as talc-calcium carbonate costructures or talc-titanium dioxide costructures, or their blends.

Preferentially they are chosen from among the natural or synthetic calcium carbonates or talc or kaolin or their blends.

In a very preferential manner they are chosen from among the natural or synthetic calcium carbonates or their blends.

These aqueous dispersions of mineral matter are also characterised in that they contain between 0.1% and 3.0%, and preferentially between 0.5% and 1.5%, by dry weight relative to the dry weight of mineral matter, of at least one homopolymer of acrylic acid.

These aqueous dispersions of mineral matter are also characterised in that they contain between 0.01% and 0.5%, and preferentially between 0.05% and 0.25%, by dry weight relative to the dry weight of mineral matter, of at least one fluorinated mineral compound.

These aqueous dispersions of mineral matter are also characterised in that the fluorinated mineral compound is chosen from among the compounds NaF, NaHF₂, H₂SiF₆, HKF₂, FeF₂, PbF₂, HNH₄F₂ and their blends, and preferentially from among the compounds NaF, H₂SiF₆, HKF₂, and their blends, and in that it is preferentially the compound NaF.

These aqueous dispersions of mineral matter are also characterised in that the homopolymer of acrylic acid used is neutralised, totally or partially, by a neutralisation agent chosen from among the hydroxides and/or oxides of calcium or magnesium, the hydroxides of sodium, potassium or ammonium, or their blends, preferentially by a neutralisation agent chosen from among sodium or ammonium hydroxide, or their blends, and very preferentially by a neutralisation agent which is ammonium hydroxide.

These aqueous dispersions of mineral matter are also characterised in that the homopolymer of acrylic acid used has an average molecular mass of between 1,000 and 150,000 Daltons, preferentially between 5,000 and 100,000 Daltons, and more preferentially between 15,000 and 80,000 Daltons.

These aqueous dispersions of mineral matter are also characterised in that the homopolymer of acrylic acid has a neutralisation rate, expressed as a molar percentage of neutralised acid sites, of preferentially between 10 and 100 Daltons, between 50 and 100 Daltons, and more preferentially between 70 and 100.

Another object of the invention lies in the use of these aqueous dispersions in the fields of manufacture of aqueous formulations containing mineral matter, notably in the paper field, and more particularly in the manufacture of paper sheet and in the manufacture of paper coatings intended for the production of a coated sheet of paper, in the plastic and paint sectors, and also in cosmetics, and more particularly in the manufacture of dental pastes.

The following example illustrates the invention without however limiting its scope.

### EXAMPLE 1

This example illustrates the use of dispersing agents in the following process:
(a) preparation of an aqueous suspension of calcium carbonate, which is a Norwegian marble, the granulometry of which is such that 75% by weight of the particles have a diameter of less than 1 μm (measured by a device of the Sedigraph™ 5100 type sold by the company MICROMERITICS™), by grinding of the said carbonate without a grinding aid agent and without a dispersing agent, at a concentration by dry weight of mineral matter equal to 20% of the total weight of the said suspension,
(b) followed by concentration of the aqueous suspension of calcium carbonate thus obtained during stage a) by means of a thermal evaporator, with a view to obtaining as high as possible a concentration by dry weight of mineral matter,
   in which during stage b) the following have been used:
either a dispersing agent according to the invention in the form of a combination:
   of at least one homopolymer of acrylic acid, totally or partially neutralised,
   and of at least one fluorinated mineral compound. or a dispersing agent according to the prior art.

For each of the tests No. 1 to 17 for each aqueous dispersion of mineral matter, according to the methods well known to the skilled man in the art, the Brookfield™ viscosity at 100 RPM was determined immediately after stage b), at 25° C., and noted μ₁₀₀^{t0}.

### Test No. 1

This test illustrates the invention and uses, relative to the dry weight of calcium carbonate:
0.75% by dry weight of a homopolymer of acrylic acid, 70% of which by mole of the acid sites are neutralised by sodium hydroxide, of molecular weight M_{w} equal to 13,300 Daltons, and having a viscosity index (as measured according to the method described in document WO 01/048 093) equal to 0.075,
and 0.10% by dry weight of sodium fluoride.

### Test No. 2

This test illustrates the invention and uses, relative to the dry weight of calcium carbonate:
0.75% by dry weight of a homopolymer of acrylic acid totally neutralised by ammonium hydroxide, and of molecular weight M_{w} equal to 10,000 Daltons, and having a viscosity index (as measured according to the method described in document WO 01/048 093) equal to 0.07,
and 0.10% by dry weight of sodium fluoride.

### Test No. 3

This test illustrates the invention and uses, relative to the dry weight of calcium carbonate:
0.75% by dry weight of a homopolymer of acrylic acid, 50% of which by mole of the acid sites are neutralised by sodium hydroxide and 15% of which by mole of the acid sites are neutralised by magnesium hydroxide, of molecular weight M_{w} equal to 10,000 Daltons, and having a viscosity index (as measured according to the method described in document WO 01/048 093) equal to 0.07,
and 0.10% by dry weight of sodium fluoride.

### Test No. 4

This test illustrates the invention and uses, relative to the dry weight of calcium carbonate:
0.80% by dry weight of a homopolymer of acrylic acid, 40% of which by mole of the acid sites are neutralised by sodium hydroxide, of molecular weight M_{w} equal to 10,000 Daltons, and having a viscosity index (as measured according to the method described in document WO 01/048 093) equal to 0.07,
and 0.10% by dry weight of sodium fluoride.

### Test No. 5

This test illustrates the invention and uses, relative to the dry weight of calcium carbonate:
0.70% by dry weight of a homopolymer of acrylic acid, 80% of which by mole of the acid sites are neutralised by sodium hydroxide, of molecular weight M_{w} equal to 10,000 Daltons, and having a viscosity index (as measured according to the method described in document WO 01/048 093) equal to 0.07,
and 0.10% by dry weight of sodium fluoride.

### Test No. 6

This test illustrates the invention and uses, relative to the dry weight of calcium carbonate:
0.70% by dry weight of a homopolymer of acrylic acid, 60% of which by mole of the acid sites are neutralised by sodium hydroxide, of molecular weight M_{w} equal to 10,000 Daltons, and having a viscosity index (as measured according to the method described in document WO 01/048 093) equal to 0.07,
and 0.10% by dry weight of sodium fluoride.

### Test No. 7

This test illustrates the invention and uses, relative to the dry weight of calcium carbonate:
0.75% by dry weight of a homopolymer of acrylic acid, 15% of which by mole of the acid sites are neutralised by sodium hydroxide, of molecular weight M_{w} equal to 10,000 Daltons, and having a viscosity index (as measured according to the method described in document WO 01/048 093) equal to 0.07,
and 0.10% by dry weight of sodium fluoride.

### Test No. 8

This test illustrates the invention and uses, relative to the dry weight of calcium carbonate:
0.73% by dry weight of a homopolymer of acrylic acid, 40% of which by mole of the acid sites are neutralised by sodium hydroxide, of molecular weight M_{w} equal to 10,000 Daltons, and having a viscosity index (as measured according to the method described in document WO 01/048 093) equal to 0.07,
and 0.05% by dry weight of the compound H₂SiF₆.

### Test No. 9

This test illustrates the invention and uses, relative to the dry weight of calcium carbonate:
0.70% by dry weight of a homopolymer of acrylic acid, 80% of which by mole of the acid sites are neutralised by sodium hydroxide, of molecular weight M_{w} equal to 10,000 Daltons, and having a viscosity index (as measured according to the method described in document WO 01/048 093) equal to 0.07,
and 0.25% by dry weight of the compound HKF₂.

### Test No. 10

This test illustrates the invention and uses, relative to the dry weight of calcium carbonate:
0.75% by dry weight of a homopolymer of acrylic acid, 60% of which by mole of the acid sites are neutralised by sodium hydroxide, of molecular weight M_{w} equal to 10,000 Daltons, and having a viscosity index (as measured according to the method described in document WO 01/048 093) equal to 0.07,
and 0.25% by dry weight of the compound HKF₂.

### Test No. 11

This test illustrates the invention and uses, relative to the dry weight of calcium carbonate:
0.70% by dry weight of a homopolymer of acrylic acid, 80% of which by mole of the acid sites are neutralised by sodium hydroxide, of molecular weight M_{w} equal to 10,000 Daltons, and having a viscosity index (as measured according to the method described in document WO 01/048 093) equal to 0.07,
and 0.25% by dry weight of the compound HKF₂.

### Test No. 12

This test illustrates the invention and uses, relative to the dry weight of calcium carbonate:
0.70% by dry weight of a homopolymer of acrylic acid, 80% of which by mole of the acid sites are neutralised by sodium hydroxide, of molecular weight M_{w} equal to 10,000 Daltons, and having a viscosity index (as measured according to the method described in document WO 01/048 093) equal to 0.07,
and 0.25% by dry weight of the compound PbF₂.

### Test No. 13

This test illustrates the invention and uses, relative to the dry weight of calcium carbonate:
0.70% by dry weight of a homopolymer of acrylic acid, 40% of which by mole of the acid sites are neutralised by sodium hydroxide, of molecular weight M_{w} equal to 10,000 Daltons, and having a viscosity index (as measured according to the method described in document WO 01/048 093) equal to 0.07,
and 0.25% by dry weight of the compound HNH₄F₂

### Test No. 14

This test illustrates the invention and uses, relative to the dry weight of calcium carbonate:
0.75% by dry weight of a homopolymer of acrylic acid totally neutralised by sodium hydroxide, of molecular weight M_{w} equal to 50,000 Daltons, and having a viscosity index equal to 0.8 (as measured according to the method described in document WO 01/048 093),
and 0.10% by dry weight of sodium fluoride.

### Test No. 15

This test illustrates the invention and uses, relative to the dry weight of calcium carbonate:
0.75% by dry weight of a homopolymer of acrylic acid totally neutralised by potassium hydroxide, of molecular weight M_{w} equal to 50,000 Daltons, and having a viscosity index equal to 0.8 (as measured according to the method described in document WO 01/048 093),
and 0.10% by dry weight of sodium fluoride.

### Test No. 16

This test illustrates the invention and uses, relative to the dry weight of calcium carbonate:
0.75% by dry weight of a homopolymer of acrylic acid, 50% of which by mole of the acid sites are neutralised by magnesium hydroxide and 30% of which by mole of the acid sites are neutralised by sodium hydroxide, of molecular weight M_{w} equal to 50,000 Daltons, and having a viscosity index equal to 0.8 (as measured according to the method described in document WO 01/048 093),
and 0.10% by dry weight of sodium fluoride.

### Test No. 17

This test illustrates the invention and uses, relative to the dry weight of calcium carbonate:
0.75% by dry weight of a homopolymer of acrylic acid, 80% of which by mole of the acid sites are neutralised by sodium hydroxide, of molecular weight M_{w} equal to 50,000 Daltons, and having a viscosity index equal to 0.8 (as measured according to the method described in document WO 01/048 093),
and 0.10% by dry weight of sodium fluoride.

For each of the tests No. 1 to 17, the values of the Brookfield™ viscosity measured at t=0, at 25° C., and at a rotational speed of 100 RPM (in mPa·s) noted μ₁₀₀^{t0}, and the values of the dry extract (as a percentage by dry weight of mineral matter relative to the total weight of the dispersion obtained), noted DE, are indicated in table 1.

TABLE 1
characteristics of the dispersants according to the invention and values of
DE and μ₁₀₀^{t0} for the dispersions obtained according to the invention.

| | dispersant | | | | | | | |
|---|---|---|---|---|---|---|---|---|
| | homopolymer of acrylic acid | | | | fluorinated | | | |
| | | neut. | molecular | | compound | | dispersion | |
| test | neut. | rate¹ | weight | quantity² | | quantity³ | μ₁₀₀^{t0} | DE |
| NO. | agent | (%) | (Daltons) | (%) | nature | (%) | (mPa · s) | (%) |
| 1 | NaOH | 70 | 13,300 | 0.75 | NaF | 0.10 | 860 | 71.3 |
| 2 | NH₃ | 100 | 10,000 | 0.75 | NaF | 0.10 | 140 | 71.5 |
| 3 | NaOH | 50 | 10,000 | 0.75 | NaF | 0.10 | 1340 | 65.4 |
| | Mg(OH)₂ | 15 | | | | | | |
| 4 | NaOH | 40 | 10,000 | 0.80 | NaF | 0.10 | 4,900 | 68.3 |
| 5 | NaOH | 80 | 10,000 | 0.70 | NaF | 0.10 | 2,240 | 65.8 |
| 6 | NaOH | 60 | 10,000 | 0.70 | NaF | 0.10 | 2,420 | 70.0 |
| 7 | NaOH | 15 | 10,000 | 0.75 | NaF | 0.10 | 245 | 70.1 |
| 8 | NaOH | 40 | 10,000 | 0.73 | H₂SiF₆ | 0.05 | 2,470 | 66.0 |
| 9 | NaOH | 80 | 10,000 | 0.70 | HKF₂ | 0.25 | 2,460 | 66.5 |
| 10 | NaOH | 60 | 10,000 | 0.75 | HKF₂ | 0.25 | 2,410 | 65.4 |
| 11 | NaOH | 80 | 10,000 | 0.70 | FeF₂ | 0.25 | 1,500 | 71.0 |
| 12 | NaOH | 80 | 10,000 | 0.70 | PbF₂ | 0.25 | 2,450 | 65.5 |
| 13 | NaOH | 40 | 10,000 | 0.70 | HNH₄F₂ | 0.25 | 3,290 | 65.5 |
| 14 | NaOH | 100 | 50,000 | 0.75 | NaF | 0.10 | 245 | 70.1 |
| 15 | KOH | 100 | 50,000 | 0.75 | NaF | 0.10 | 555 | 70.7 |
| 16 | Mg(OH)₂ | 50 | 50,000 | 0.75 | NaF | 0.10 | 860 | 70.0 |
| | NaOH | 30 | | | | | | |
| 17 | NaOH | 80 | 50,000 | 0.75 | NaF | 0.10 | 620 | 71.1 |

In this table:
neut. rate¹ (%) designates the neutralisation rate of each homopolymer, expressed as a molar percentage of neutralised acid sites,
quantity² designates the quantity of homopolymer used, expressed as a percentage by dry weight of the said polymer relative to the total dry weight of mineral matter,
quantity³ designates the quantity of fluorinated mineral compound used, expressed as a percentage by dry weight of the said fluorinated mineral compound relative to the total dry weight of mineral matter,
μ₁₀₀^{t0} designates the Brookfield ™ viscosity measured at 100 RPM immediately after stage b) and noted μ₁₀₀^{t0},
DE designates the dry extract expressed as a percentage by dry weight of mineral matter relative to the total weight of each dispersion.

A reading of table 1 therefore demonstrates that the use of dispersants according to the invention enables the initial suspensions of calcium carbonate which had been ground without a dispersing agent or grinding aid agent at a dry matter concentration of less than 40% of the total weight of the said suspension to be dispersed efficiently and at concentrations by dry weight of mineral matter greater than 65% of the total weight of the said dispersion.

In an even more favourable manner, this table demonstrates that the use of dispersants according to the invention enables, in the process described hereabove, aqueous dispersions of calcium carbonate to be obtained with concentrations by dry weight of mineral matter greater than 65%, and an immediate Brookfield™ viscosity measured at 100 RPM less than 5,000 mPa·s.

For some tests, in a surprising manner, dry weight concentrations of mineral matter greater than 70%, and an immediate Brookfield™ viscosity measured at 100 RPM less than 500 mPa·s, are even obtained (case of tests No. 2, 7 and 14).

Such results respond perfectly to the demand of the skilled man in the art.

### Test No. 18

This test illustrates the prior art and uses 0.75% by dry weight, relative to the dry weight of calcium carbonate, of a homopolymer of acrylic acid totally neutralised by sodium hydroxide, and of molecular weight equal to 14,000 Daltons.

### Test No. 19

This test illustrates the prior art and uses 0.75% by dry weight, relative to the dry weight of calcium carbonate, of a homopolymer of acrylic acid which has been totally neutralised by sodium hydroxide, and of molecular weight equal to 12,000 Daltons.

### Test No. 20

This test illustrates the prior art and uses 0.75% by dry weight, relative to the dry weight of calcium carbonate, of a homopolymer of acrylic acid which has been totally neutralised by ammonium hydroxide, and of molecular weight equal to 10,000 Daltons.
DE designates the dry extract expressed as a percentage by dry weight of mineral matter relative to the total weight of each dispersion.
   A reading of table 2 demonstrates that none of the tests No. 18 to 22 enables a Brookfield viscosity—measured at 100 RPM immediately after stage b)—less than 5,000 mPa·s, and simultaneously a dry extract greater than 65% by weight of mineral matter, to be obtained: this however remains the principal goal of the skilled man in the art through the technical problem which the present Application seeks to resolve.

A comparison with the results shown in table 1 therefore demonstrates the surprising effect obtained through the use of the polymers according to the invention.

Lastly, a test was undertaken using a copolymer of acrylic acid and of maleic anhydride as described in document EP 0 850 685 (test No. 23), and a test using a homopolymer of acrylic acid as described in document WO 01/048 093 (test No. 24): these two tests illustrate the prior art and use the said polymers in the same process as that described for tests No. 1 to 17.

### Test No. 23

This test illustrates the prior art and uses, relative to the dry weight of calcium carbonate, 0.75% by dry weight of a copolymer of acrylic acid and of maleic anhydride (in a 3:1 molar blend), totally neutralised by soda, and of molecular weight M_{w} equal to 22,500 Daltons.

### Test No. 24

This test illustrates the prior art and uses, relative to the dry weight of calcium carbonate, 0.75% by dry weight of a homopolymer of acrylic acid totally neutralised by soda, and of molecular weight M_{w} equal to 50,000 Daltons, and having a viscosity index (as measured according to the method described in document WO 01/048 093) equal to 0.8.

For each of the tests No. 23 and 24, the values of the Brookfield™ viscosity measured at t=0, at 25° C., and at a rotational speed of 100 RPM (in mPa·s) noted μ₁₀₀^{t0}, and the values of the dry extract (as a percentage by dry weight of mineral matter relative to the total weight of the dispersion obtained), noted DE, are indicated in table 3.

### Test No. 21

This test illustrates the prior art and uses 0.80% by dry weight, relative to the dry weight of calcium carbonate, of a homopolymer of acrylic acid which has been totally neutralised by ammonium hydroxide, and of molecular weight equal to 10,000 Daltons.

### Test No. 22

This test illustrates the prior art and uses 0.85% by dry weight, relative to the dry weight of calcium carbonate, of a homopolymer of acrylic acid which has been totally neutralised by ammonium hydroxide, and of molecular weight equal to 10,000 Daltons.

For each of the tests No. 18 to 22, the values of the Brookfield™ viscosity measured at t=0, at 25° C., and at a rotational speed of 100 RPM (in mPa·s) noted μ₁₀₀^{t0}, and the values of the dry extract (as a percentage by dry weight of mineral matter relative to the total weight of the dispersion obtained), noted DE, are indicated in table 2.

TABLE 2
characteristics of the dispersants according to the prior art
and values of DE and μ₁₀₀^{t0} for the dispersions obtained.

| | Dispersant | | | | | |
|---|---|---|---|---|---|---|
| | (homopolymer of acrylic acid) | | | | | |
| | | neut. | molecular | | Dispersion | |
| test | neut. | rate¹ | weight | quantity² | μ₁₀₀^{t0} | DE |
| NO. | agent | (%) | (Daltons) | (%) | (mPa · s) | (%) |
| 18 | NaOH | 100 | 14,000 | 0.75 | 9,200 | 65.9 |
| 19 | NaOH | 100 | 12,000 | 0.75 | 5,500 | 64.9 |
| 20 | NH₃ | 100 | 10,000 | 0.75 | 5,600 | 40.0 |
| 21 | NH₃ | 100 | 10,000 | 0.80 | 5,500 | 45.1 |
| 22 | NH₃ | 100 | 10,000 | 0.85 | 6,230 | 55.2 |

In this table:
neut. rate¹ (%) designates the neutralisation rate of each homopolymer, expressed as a molar percentage of neutralised acid sites,
quantity² designates the quantity of homopolymer used, expressed as a percentage by dry weight of the said polymer relative to the total dry weight of mineral matter,
μ₁₀₀^{t0} designates the Brookfield ™ viscosity measured at 100 RPM immediately after stage b) and noted μ₁₀₀^{t0},
DE designates the dry extract expressed as a percentage by dry weight of mineral matter relative to the total weight of each dispersion.

The results obtained for tests No. 2, 7 and 14 are also shown in table 3.

TABLE 3
characteristics of the dispersants according to the invention and values of DE
and μ₁₀₀^{t0} for the dispersions obtained according to the invention.

| | | dispersing agent | | | | | | | | |
|---|---|---|---|---|---|---|---|---|---|---|
| | | | | | | | fluorinated | | | |
| | | | | neut.¹ | molecular | | compound | | dispersion | |
| test | invention/ | | neut. | rate | weight | quantity² | | quantity³ | μ₁₀₀^{t0} | DE |
| N° | prior art | nature | agent | (%) | (Daltons) | (%) | nature | (%) | (mPa · s) | (%) |
| 23 | prior art | copolymer | NaOH | 100 | 22,500 | 0.75 | — | — | 260 | 70.1 |
| 24 | prior art | homopolymer | NaOH | 100 | 50,000 | 0.75 | — | — | 5,270 | 71.8 |
| 2 | invention | homopolymer | NH₃ | 100 | 10,000 | 0.75 | NaF | 0.1 | 140 | 71.5 |
| 7 | invention | homopolymer | NaOH | 15 | 10,000 | 0.75 | NaF | 0.1 | 245 | 70.1 |
| 14 | invention | homopolymer | NaOH | 100 | 50,000 | 0.75 | NaF | 0.1 | 245 | 70.1 |

In this table:
the term homopolymer designates a homopolymer of acrylic acid,
the term copolymer designates a copolymer of acrylic acid and of maleic anhydride (in a 3:1 molar blend) and of molecular weight 22,500 Daltons,
neut. rate¹ (%) designates the neutralisation rate of each homopolymer or copolymer, expressed as a molar percentage of neutralised acid sites,
quantity² designates the quantity of homopolymer or of copolymer used, expressed as a percentage by dry weight of the said polymer relative to the total dry weight of mineral matter,
quantity³ designates the quantity of fluorinated mineral compound used, expressed as a percentage by dry weight of the said fluorinated mineral compound relative to the total dry weight of mineral matter,
μ₁₀₀^{t0} designates the Brookfield ™ viscosity measured at 100 RPM immediately after stage b) and noted μ₁₀₀^{t0},
DE designates the dry extract expressed as a percentage by dry weight of mineral matter relative to the total weight of each dispersion.

These results demonstrate that the use of the new dispersants according to the invention allow, after their optimisation, the skilled man in the art to be supplied with even more efficient solutions than those consisting of the very restrictive selections proposed in the only two documents of the state of the technique previously accessible to them to resolve their problem (WO 01/048 093 and EP 0 850 685).

### EXAMPLE 2

This example illustrates the use of dispersing agents in the following process:
(a) preparation of an aqueous suspension of calcium carbonate, which is a Norwegian marble, the granulometry of which is such that 75% by weight of the particles have a diameter of less than 1 μm (measured by a device of the Sedigraph™ 5100 type sold by the company MICROMERITICS™), by grinding of the said carbonate without a grinding aid agent and without a dispersing agent, at a concentration by dry weight of mineral matter equal to 30% of the total weight of the said suspension,
(b) followed by concentration of the aqueous suspension of calcium carbonate thus obtained during stage a) by means of a thermal evaporator, with a view to obtaining as high as possible a concentration by dry weight of mineral matter,
   where a dispersing agent according to the invention has been used in the form of a combination:
of at least one homopolymer of acrylic acid, totally or partially neutralised,
and of at least one fluorinated mineral compound.

For each of the following tests, for each aqueous dispersion of mineral matter, according to the methods well known to the skilled man in the art, the Brookfield™ viscosity at 100 RPM was determined immediately after stage b), at 25° C., and noted μ₁₀₀^{t0}.

### Test No. 25

This test illustrates the invention and uses, relative to the dry weight of calcium carbonate:
0.10% by dry weight of NaHF₂, introduced between stage a) and stage b),
and 0.60% by dry weight of a homopolymer of acrylic acid, all the acid sites of which are neutralised by sodium hydroxide, and of molecular weight M_{w} equal to 10,000 Daltons, introduced in 3 doses during stage b).

### Test No. 26

This test illustrates the invention and uses, relative to the dry weight of calcium carbonate:
0.10% by dry weight of NaF and 0.1% by dry weight of H₂SiF₆, introduced between stage a) and stage b),
and 0.60% by dry weight of a homopolymer of acrylic acid, all the acid sites of which are neutralised by sodium hydroxide, and of molecular weight M_{w} equal to 10,000 Daltons, introduced during and after stage b).

TABLE 4
characteristics of the dispersants according to the invention and values of
DE and μ₁₀₀^{t0} for the dispersions obtained according to the invention.

| | dispersant | | | | | | | |
|---|---|---|---|---|---|---|---|---|
| | homopolymer of acrylic acid | | | | fluorinated | | | |
| | | neut. | molecular | | compound | | dispersion | |
| test | neut. | rate¹ | weight | quantity² | | quantity³ | μ₁₀₀^{t0} | DE |
| No. | agent | (%) | (Daltons) | (%) | nature | (%) | (mPa · s) | (%) |
| 25 | NaOH | 100 | 10,300 | 0.60 | NaHF₂ | 0.10 | 4520 | 70.2 |
| 26 | NaOH | 100 | 10,300 | 0.60 | NaHF₂ + | 0.10 + | 4900 | 70.1 |
| | | | | | H₂SiF₆ | 0.10 | | |

Table 4 uses the abbreviations of table 1; their meanings are given above.

A reading of table 4 therefore demonstrates that the use of dispersants according to the invention enables the initial suspensions of calcium carbonate which had been ground without a dispersing agent or grinding aid agent at a dry matter concentration of less than 40% of the total weight of the said suspension to be dispersed efficiently and at concentrations by dry weight of mineral matter greater than 65% of the total weight of the said dispersion.

In an even more favourable manner, this table demonstrates that the use of dispersants according to the invention enables, in the process described hereabove, aqueous dispersions of calcium carbonate to be obtained with concentrations by dry weight of mineral matter greater than 65%, and an immediate Brookfield™ viscosity measured at 100 RPM less than 5,000 mPa·s.

### EXAMPLE 3

This example illustrates the use of dispersing agents in the following process:
(a) preparation of an aqueous suspension of calcium carbonate, which is a Norwegian marble, the granulometry of which is such that 75% by weight of the particles have a diameter of less than 1 μm (measured by a device of the Sedigraph™ 5100 type sold by the company MICROMERITICS™), by grinding of the said carbonate without a grinding aid agent and without a dispersing agent, at a concentration by dry weight of mineral matter equal to 16% of the total weight of the said suspension,
(b) followed by concentration of the aqueous suspension of calcium carbonate thus obtained during stage a) by means of a thermal evaporator, with a view to obtaining as high as possible a concentration by dry weight of mineral matter,
   where a dispersing agent according to the invention has been used in the form of a combination:
of at least one homopolymer of acrylic acid, totally or partially neutralised,
and of at least one fluorinated mineral compound.

### Test No. 27

This test illustrates the invention and uses, relative to the dry weight of calcium carbonate:
0.10% by dry weight of NaF, introduced between stage a) and stage b),
and 0.80% by dry weight of a homopolymer of acrylic acid, obtained by the controlled radical polymerisation method known as RAFT (according to the method described in document 2 821 620), all the acid sites of which are neutralised by sodium hydroxide, and of molecular weight M_{w} equal to 9,500 Daltons, introduced during stage b), and then 0.2% introduced after stage b).

The Applicant indicates that all the homopolymers of acrylic acid used in tests No. 1 to 26 were obtained by traditional polymerisation methods.

TABLE 5
characteristics of the dispersant according to the invention and value of
DE and μ₁₀₀^{t0} for the dispersion obtained according to the invention.

| | dispersant | | | | | | | |
|---|---|---|---|---|---|---|---|---|
| | homopolymer of acrylic acid | | | | fluorinated | | | |
| | | neut. | molecular | | compound | | dispersion | |
| test | neut. | rate¹ | weight | quantity² | | quantity³ | μ₁₀₀^{t0} | ES |
| No. | agent | (%) | (Daltons) | (%) | nature | (%) | (mPa · s) | (%) |
| 27 | NaOH | 100 | 9500 | 1.00 | NaF | 0.10 | 4350 | 65.1 |

A reading of table 5 demonstrates that the use of the dispersant according to the invention enables an initial suspension of calcium carbonate which had been ground without a dispersing agent or grinding aid agent at a dry matter concentration of less than 40% of the total weight of the said suspension to be dispersed efficiently and at dry weight concentrations of mineral matter greater than 65% of the total weight of the said dispersion.

In an even more favourable manner, this table demonstrates that the use of the dispersant according to the invention enables, in the process described hereabove, aqueous dispersions of calcium carbonate to be obtained with concentrations by dry weight of mineral matter greater than 65%, and an immediate Brookfield™ viscosity measured at 100 RPM less than 5,000 mPa·s.

Finally, the Applicant indicates that all the fluorinated compounds were used in connection with the invention in the form of a solution, except for H₂SiF₆, which had been used in the form of an aqueous solution.

1. A process for the manufacture of an aqueous dispersion of mineral matter, comprising the stages of:
(a) preparing an aqueous suspension of mineral matter by grinding of the said mineral matter without a dispersing agent and/or grinding aid agent, where the said suspension has a dry weight concentration of mineral matter less than or equal to 40% of its total weight,
(b) concentrating the aqueous suspension of mineral matter obtained during stage a) by mechanical and/or thermal means, with a view to obtaining a concentration by dry weight of mineral matter at least equal to 65% of the total weight of the said dispersion,
characterised in that:
at least one dispersing agent is introduced between stage a) and stage b), and/or during stage b), and/or during and after stage b)
in the form of a combination:
of at least one homopolymer of acrylic acid,
and of at least one fluorinated mineral compound.

2. The process according to claim 1, characterised in that the aqueous suspension of mineral matter obtained during stage a) has a concentration by dry weight of mineral matter preferentially less than 35%, and more preferentially less than 30%, (relative to the total weight of the said suspension) and in that the aqueous dispersion of mineral matter obtained after stage b) has a concentration by dry weight of mineral matter preferentially greater than 68%, and more preferentially greater than 70% (relative to the total weight of the said dispersion).

3. The process according to claim 1, characterised in that the aqueous dispersion of mineral matter obtained immediately after stage b) has a Brookfield™ viscosity measured at 100 RPM of less than 5,000 mPa·s, preferentially less than 2,000 mPa·s, very preferentially less than 1,000 mPa·s, and extremely preferentially less than 500 mPa·s.

4. The process according to claim 1, characterised in that the mineral matter is chosen from among natural or synthetic calcium carbonate, the dolomites, kaolin, talc, cement, gypsum, lime, magnesia, titanium dioxide, satin white, aluminium trioxide, or again aluminium trihydroxide, the silicas, mica and a blend of these fillers one with another, such as talc-calcium carbonate blends, calcium carbonate-kaolin blends, or again blends of calcium carbonate with aluminium trihydroxide or aluminium trioxide, or again blends with synthetic or natural fibres, or again mineral costructures such as talc-calcium carbonate costructures or talc-titanium dioxide costructures, or their blends.

5. The process according to claim 4, characterised in that the mineral matter is chosen from among the natural or synthetic calcium carbonates or talc or kaolin or their blends.

6. The process according to claim 5, characterised in that the mineral matter is chosen from among the natural or synthetic calcium carbonates or their blends.

7. The process according to claim 1, characterised in that the fluorinated mineral compound on the one hand, and the homopolymer of acrylic acid on the other hand, are introduced simultaneously, or in that the fluorinated mineral compound is firstly introduced, and subsequently the homopolymer of acrylic acid, or firstly the homopolymer of acrylic acid, and subsequently the fluorinated mineral compound.

8. The process according to claim 1, characterised in that the fluorinated mineral compound and the homopolymer of acrylic acid are introduced in the form of an aqueous suspension and/or an aqueous solution when they are introduced simultaneously.

9. The process according to claim 1, characterised in that the fluorinated mineral compound is introduced in the form of powder and/or in the form of an aqueous suspension and/or in the form of an aqueous solution, and in that the homopolymer of acrylic acid is introduced in the form of an aqueous solution when these two compounds are introduced one after the other, whatever the order of introduction.

10. The process according to claim 1, characterised in that between 0.1% and 3.0%, and preferentially between 0.5% and 1.5%, by dry weight relative to the dry weight of mineral matter, of at least one homopolymer of acrylic acid is used.

11. The process according to claim 1, characterised in that between 0.01% and 0.5%, and preferentially between 0.05% and 0.25%, by dry weight relative to the dry weight of mineral matter, of at least one fluorinated mineral compound is used.

12. The process according to claim 1, characterised in that the fluorinated mineral compound is chosen from among the compounds NaF, NaHF₂, H₂SiF₆, HKF₂, FeF₂, PbF₂, HNH₄F₂ and their blends.

13. The process according to claim 12, characterised in that the fluorinated mineral compound is chosen from among the compounds NaF, H₂SiF₆, HKF₂ and their blends.

14. The process according to claim 13, characterised in that the fluorinated mineral compound is the compound NaF.

15. The process according to claim 1, characterised in that the homopolymer of acrylic acid used is neutralised, totally or partially, by a neutralisation agent chosen from among the hydroxides and/or oxides of calcium or magnesium, the hydroxides of sodium, potassium, or their blends.

16. The process according to claim 15, characterised in that the neutralisation agent is chosen from among sodium hydroxide, ammonium hydroxide and their blends.

17. The process according to claim 16, characterised in that the neutralisation agent is ammonium hydroxide.

18. The process according to claim 1, characterised in that the homopolymer of acrylic acid used has an average molecular mass of between 1,000 and 150,000 Daltons.

19. The process according to claim 18, characterised in that the homopolymer of acrylic acid used has an average molecular mass of between 5,000 and 100,000 Daltons.

20. The process according to claim 19, characterised in that the homopolymer of acrylic acid used has an average molecular mass of between 15,000 and 80,000 Daltons.

21. The process according to claim 1, characterised in that the homopolymer of acrylic acid has a neutralisation rate, expressed as a molar percentage of the neutralised acid sites, of between 10 and 100.

22. The process according to claim 21, characterised in that the neutralisation rate is preferentially, expressed as a molar percentage of the neutralised acid sites, between 50 and 100.

23. The process according to claim 22, characterised in that the neutralisation rate is preferentially, expressed as a molar percentage of the neutralised acid sites, between 70 and 100.

24. Aqueous dispersions of mineral matter characterised in that they are obtained by the process according to claim 1.

25. Aqueous dispersions of mineral matter, characterised in that they contain:
at least one homopolymer of acrylic acid,
and at least one fluorinated mineral compound.

26. Aqueous dispersions of mineral matter according to claim 25, characterised in that they have a concentration by dry weight of mineral matter preferentially greater than 68%, and more preferentially greater than 70%.

27. Aqueous dispersions of mineral matter according to claim 25, characterised in that the mineral matter is chosen from among natural or synthetic calcium carbonate, the dolomites, kaolin, talc, cement, gypsum, lime, magnesia, titanium dioxide, satin white, aluminium trioxide, or again aluminium trihydroxide, the silicas, mica and a blend of these fillers one with another, such as talc-calcium carbonate blends, calcium carbonate-kaolin blends, or again blends of calcium carbonate with aluminium trihydroxide or aluminium trioxide, or again blends with synthetic or natural fibres, or again mineral costructures such as talc-calcium carbonate costructures or talc-titanium dioxide costructures, or their blends.

28. Aqueous dispersions of mineral matter according to claim 27, characterised in that the mineral matter is chosen from among the natural or synthetic calcium carbonates or talc or kaolin or their blends.

29. Aqueous dispersions of mineral matter according to claim 28, characterised in that the mineral matter is chosen from among the natural or synthetic calcium carbonates or their blends.

30. Aqueous dispersions of mineral matter according to claim 25, characterised in that they contain between 0.1% and 3.0%, and preferentially between 0.5% and 1.5%, by dry weight relative to the dry weight of mineral matter, of at least one homopolymer of acrylic acid.

31. Aqueous dispersions of mineral matter according to claim 25, characterised in that they contain between 0.01% and 0.5%, and preferentially between 0.05% and 0.25%, by dry weight relative to the dry weight of mineral matter, of at least one fluorinated mineral compound.

32. Aqueous dispersions of mineral matter according to claim 25, characterised in that the fluorinated mineral compound is chosen from among the compounds NaF, NaHF₂, H₂SiF₆, HKF₂, FeF₂, PbF₂, HNH₄F₂ and their blends.

33. Aqueous dispersions of mineral matter according to claim 32, characterised in that the fluorinated mineral compound is chosen from among the compounds NaF, H₂SiF₆, HKF₂ and their blends.

34. Aqueous dispersions of mineral matter according to claim 33, characterised in that the fluorinated mineral compound is the compound NaF.

35. Aqueous dispersions of mineral matter according to claim 25, characterised in that the homopolymer of acrylic acid used is neutralised, totally or partially, by a neutralisation agent chosen from among the hydroxides and/or oxides of calcium or magnesium, the hydroxides of sodium, potassium, or their blends.

36. Aqueous dispersions of mineral matter according to claim 35, characterised in that the neutralisation agent is chosen from among sodium hydroxide, ammonium hydroxide and their blends.

37. Aqueous dispersions of mineral matter according to claim 36, characterised in that the neutralisation agent is ammonium hydroxide.

38. Aqueous dispersions of mineral matter according to claim 25, characterised in that the homopolymer of acrylic acid used has an average molecular mass of between 1,000 and 150,000 Daltons.

39. Aqueous dispersions of mineral matter according to claim 38, characterised in that the homopolymer of acrylic acid used has an average molecular mass of between 5,000 and 100,000 Daltons.

40. Aqueous dispersions of mineral matter according to claim 39, characterised in that the homopolymer of acrylic acid used has an average molecular mass of between 15,000 and 80,000 Daltons.

41. Aqueous dispersions of mineral matter according to claim 25, characterised in that the homopolymer of acrylic acid has a neutralisation rate, expressed as a molar percentage of the neutralised acid sites, of between 10 and 100.

42. Aqueous dispersions of mineral matter according to claim 41, characterised in that the homopolymer of acrylic acid has a neutralisation rate, expressed as a molar percentage of the neutralised acid sites, of between 50 and 100.

43. Aqueous dispersions of mineral matter according to claim 42, characterised in that the homopolymer of acrylic acid has a neutralisation rate, expressed as a molar percentage of the neutralised acid sites, of between 70 and 100.

44. Aqueous formulations manufactured with the aqueous dispersions of mineral matter according to claim 24.

45. A paper sheet or paper coating manufactured with the aqueous dispersions of mineral matter according to claim 24.

46. A plastic manufactured with the aqueous dispersions of mineral matter according to claim 24.

47. Paint comprising the aqueous dispersions of mineral matter according to claim 24.

48. Dental pastes manufactured with the aqueous dispersions of mineral matter according to claim 24.
